(19) United States
(12) Patent Application Publication
(10) Pub. No.: US 2006/0264961 A1
(43) Pub. Date: Nov. 23, 2006

(54) Surgical jig

(76) Inventor: James Hamilton Murray-Brown, Braunton (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 11/410,261

(22) Filed: Apr. 24, 2006

Related U.S. Application Data
(63) Continuation of application No. PCT/GB04/04500, filed on Oct. 25, 2004.

(30) Foreign Application Priority Data
Oct. 25, 2003 (GB) 0324932.3
Dec. 5, 2003 (GB) 0328181.3

(51) Int. Cl. A61B 17/58

(52) U.S. Cl. 606/88

(57) ABSTRACT

A surgical jig for use in performing a scarf osteotomy on the first metatarsal of the forefoot includes a cutting guide that is adapted to guide a cutting implement relative to a reference surface that is adapted to be placed against the plantar aspect of a forefoot. The jig has guide surfaces for guiding both the longitudinal cut and the transverse cuts of the operation. The jig is selected from one in a set of jigs by an overlay template, through which an X-ray of the forefoot can be viewed.

### CROSS-REFERENCE TO RELATED APPLICATIONS

This application is a continuation of International Application No. PCT/GB2004/004500, filed Oct. 25, 2004, which claims priority from U.K. Patent Application Nos. 0324932.3, filed Oct. 25, 2003 and 0328181.3, filed Dec. 5, 2003. The disclosures of all of these applications are incorporated herein by reference.

### BACKGROUND OF THE INVENTION

The present invention relates to a surgical jig, and more particularly to a surgical jig for guiding a cutting implement when operating on the human forefoot.

Hallux Valgus is a relatively common disorder of the forefoot in which the first metatarsal diverges from the second metatarsal at an angle greater than usual, typically in excess of 9 degrees and often up to 20 degrees or more. This angle is known as the intermetatarsal angle (IMA). The usual articulation between the first. metatarsal head and the two sesamoid bones beneath it is disturbed, typically causing pain as weight is put onto the foot. In addition the first metatarsal head can become displaced dorsally, typically causing weight to be transferred to the second and third metatarsal heads when walking or standing, which can be painful. Furthermore the big toe tends to become angled towards the lesser toes, crowding them. Finally, the forefoot is broad which causes difficulty with shoe fitting.

There are several known methods of surgery for treatment of Hallux Valgus, but a commonly used operation is a scarf osteotomy carried out on the first metatarsal bone.

The term scarf originates from ancient carpentry. This osteotomy is versatile, because the IMA can be reduced, the first metatarsal shortened or lengthened, and the position of the first metatarsal head altered in order to improve articulation with the sesamoid bones. The cuts of the scarf osteotomy can be made such that when the first metatarsal head is translated laterally after the osteotomy, the first metatarsal head is also moved in a plantar direction (towards the sole or plantar aspect of the forefoot), typically causing a greater proportion of weight to be borne by the first metatarsal head relative to the second and third metatarsal heads. Reducing the IMA narrows the forefoot. Very often a bunionectomy is also performed, removing excess bone from the medial aspect of the first metatarsal head.

During surgery, the first metatarsal bone is partially exposed by a medial approach, and a longitudinal cut made along the metatarsal. Two transverse cuts are then made at the ends of the longitudinal cut, so that the completed osteotomy is a Z-type form.

The slope of the longitudinal cut enables lateral and vertical translation of the first metatarsal head. In addition, the transverse cuts at each end of the longitudinal cut may be made in such planes that the first metatarsal is shortened or lengthened when the translation of the first metatarsal head is carried out. Commonly the surgeon aims to maintain the length of the great toe relative to the second toe.

The cuts are usually made with oscillating or reciprocating saw blades, and the surgeon may find it difficult to make the cuts in the desired planes in accordance with a pre-operative plan. The pre-operative plan is derived from examination of the patient's foot and radiographs (x-rays). It is also the case that most other forefoot surgery is conducted free hand.

### SUMMARY OF THE INVENTION

According to a first aspect of the present invention there is provided a surgical jig comprising a cutting guide adapted to guide a cutting implement and a reference surface adapted to be placed against the plantar aspect of a forefoot.

It is an advantage of the surgical jig that a surgeon is able to make a cut in the correct plane relative to the plantar aspect of a forefoot, in accordance with a pre-operative plan.

It is a further advantage of the invention that a surgeon is able to guide a longitudinal cut of the first metatarsal with a jig that refers to the plantar aspect of the foot, so that the resulting cut is in a known and reproducible plane.

Preferably the cutting guide includes a planar surface disposed at an angle to the reference surface.

Preferably the reference surface is the upper surface of a base plate and the cutting guide is rigidly connected to the base plate.

Preferably an angled spacer is provided for increasing the angle between the planar surface and the reference surface, the spacer being attached to the upper surface of, the base plate, and the upper surface of the spacer becoming the reference surface.

Preferably end stops are provided at one or both ends of the planar surface.

Preferably the end stops are provided at a predetermined angle relative to a longitudinal axis of the cutting guide.

Preferably the end stops are provided by abutments, the abutments having faces adjacent the planar surface and substantially perpendicular to a longitudinal axis of the cutting guide, the faces also serving as cutting guides.

Preferably one of the faces extends from the upper side of the planar surface and the other face extends from the lower side of the planar surface.

Preferably one of the faces is spaced from an end of the planar surface, enabling the cutting implement to pass the planar surface.

Preferably one or more stops are provided on the faces of the abutments.

Preferably one of the stops is provided by the planar surface.

Preferably the abutments have a curved profile on one side, which is adapted to fit against an exposed metatarsal bone.

Preferably a resilient retaining means is provided to hold the surgical jig in a fixed position relative to the plantar aspect of the forefoot.

Preferably one or more apertures are provided in the cutting guide, which enable the surgical jig to be pinned, wired or otherwise fastened to an exposed bone of the forefoot.

Preferably the surgical jig is one of a set of jigs, each having the cutting guide in a different orientation relative to the reference surface.

Preferably a template is provided for selecting a surgical jig from the set of surgical jigs.

Preferably the template comprises a transparent overlay, through which an X-ray can be viewed, the overlay having indicia thereon for comparing the orientation of the cutting guides of the jigs with the bones of the forefoot.

Preferably the template comprises a transparent overlay, through which an X-ray can be viewed, the overlay having indicia thereon for comparing the predetermined angle relative to a longitudinal axis of the cutting guide of the jigs with the bones of the forefoot.

According to a second aspect of the present invention there is provided a template for selecting a surgical jig from a set of surgical jigs, comprising a transparent overlay, through which an X-ray can be viewed, the overlay having indicia thereon for comparing the orientation of cutting guides of the jigs with the bones of the forefoot.

It is an advantage of the template that the X-ray anatomy of a forefoot can be related to the position of the cutting guide of a surgical jig, and hence the appropriate jig can be selected from a range of jigs having varying positions of cutting guide.

According to a third aspect of the present invention there is provided a jig for guiding a longitudinal saw cut in the first metatarsal bone of the foot, including a cutting block and a rigid arm extending under the plantar aspect of the foot, so that the orientation of the cutting block is referenced to the plane of the plantar aspect of the foot.

According to a fourth aspect of the present invention there is provided a jig for use during scarf osteotomy of the first metatarsal bone of the foot, including cutting blocks for the distal and proximal transverse cuts of the osteotomy and a rigid arm extending under the plantar aspect of the foot, so that the orientations of the cutting blocks are referenced to the plane of the plantar aspect of the foot.

According to a fifth aspect of the present invention there is provided a method of operating on the forefoot, comprising exposing a bone to be cut, referencing a surgical jig from the plantar aspect of the forefoot, and cutting a bone using the jig as a guide to guide a cutting tool.

For a better understanding of the present invention, and to show more clearly how it may be carried into effect, reference will now be made, by way of example, to the accompanying drawings.

### BRIEF DESCRIPTION OF THE DRAWINGS

FIG. 1 shows a perspective view of first embodiment of a surgical jig in accordance with the invention, referenced from the plantar aspect of a foot and resting against the medial aspect of the first metatarsal.

FIG. 2 shows a cross section through the surgical jig and foot shown in FIG. 1, taken on the line A-A.

FIG. 3 shows a plan view from above of the surgical jig and foot shown in FIGS. 1 and 2.

FIG. 4 shows the cuts of a scarf osteotomy on the medial aspect of the first metatarsal bone.

FIG. 5 shows a perspective view of a second embodiment of a surgical jig in accordance with the invention, referenced from the plantar aspect of the foot and resting against the medial aspect of the first metatarsal.

FIG. 6 shows a perspective view of a third and most preferred embodiment of a surgical jig in accordance with the invention, referenced from the plantar aspect of the foot and resting against the medial aspect of the first metatarsal.

FIG. 7A shows the baseplate of the surgical jig shown in FIG. 6, and one of a selection of adapter members, for changing the plane of the upper surface of the baseplate.

FIG. 7B shows the adapter member of FIG. 7A fitted to the baseplate of the surgical jig.

FIG. 8 shows an X-ray template for selecting the most suitable surgical jig from a selection of surgical jigs in accordance with the invention.

### DETAILED DESCRIPTION OF THE PREFERRED EMBODIMENT

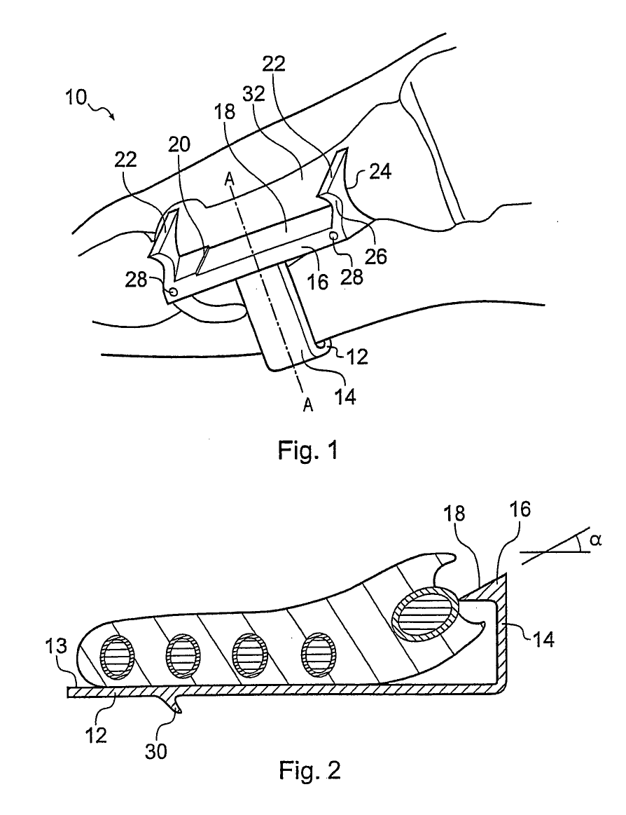

Referring firstly to FIGS. 1 and 2, a first embodiment of a surgical jig is indicated generally at 10. The jig 10 comprises a base plate 12 having an upper reference surface 13, an arm 14 extending substantially perpendicularly from one end of the base plate 12, and a cutting guide or cutting block 16 mounted to the end of the arm 14. The arm 14 and base plate 12 are integrally formed.

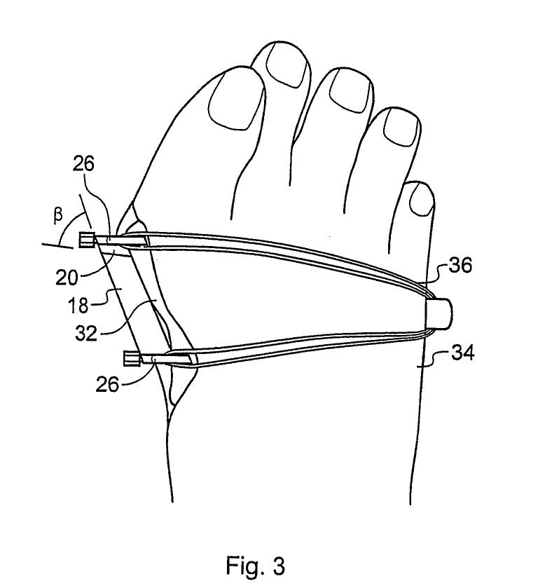

The cutting guide 16 has a planar surface 18 disposed at an angle a relative to the reference surface 13 of the base plate 12, as indicated in FIG. 2. End stops 20, one of which is shown in FIG. 1, may be provided at each end of the planar surface 18, which limit the movement of a cutting implement moving along the planar surface. The end stops are provided at an angle 0, see FIG. 3, relative to the longitudinal axis of the cutting guide.

An appendage in the form of an abutment 22 extends from each end of the cutting guide 16 and is profiled on its edge with a substantially semi-circular recess 24, which is shaped to fit against an exposed metatarsal bone. The other side of the abutment is also recessed, preferably with a V shaped recess 26 as shown in FIG. 1. An aperture 28 is provided through each end of the cutting guide 16, through which a pin or wire can be passed in order to secure the cutting guide to a first metatarsal of the foot. With reference to FIG. 2, a hook 30 is provided on the underside of the base plate 12, i.e. extending substantially in a direction opposite to that of the arm 14.

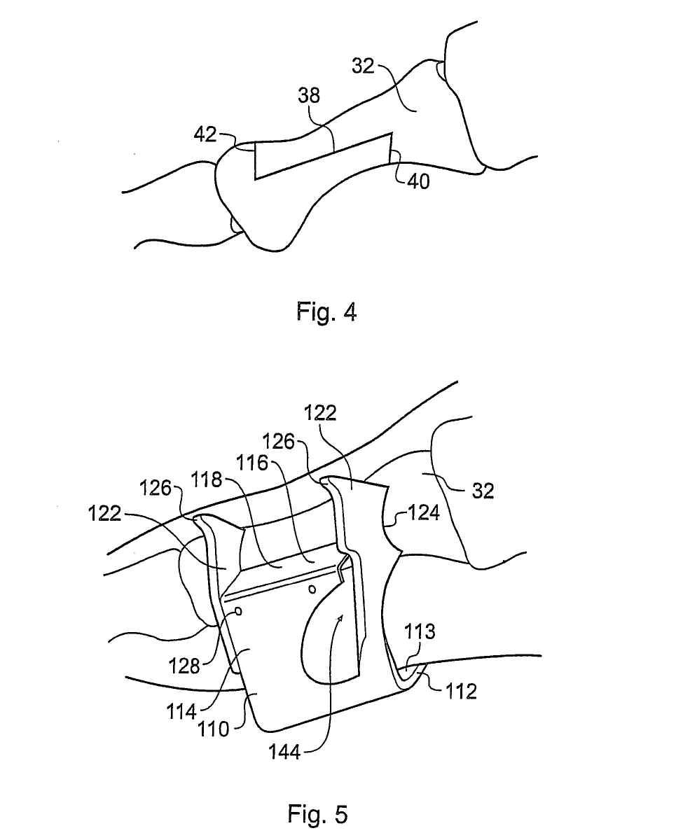

Referring now to FIG. 3, in use, a surgeon exposes the medial side of a first metatarsal 32 of a foot 34, the right foot in the Figure, and performs a bunionectomy. It is usual operative procedure to remove the bunion prior to performing a scarf osteotomy because the bunion obscures the end of the first metatarsal. When using a jig of the invention, this is also necessary to enable the jig to sit snugly against the first metatarsal, as described further below. The base plate 12 of the jig 10 is then placed against the underside of the forefoot, i.e. it is referenced against the plantar aspect of the forefoot, and the substantially semi-circular recesses 24 of the abutments 22 are placed against the side of the exposed first metatarsal 32. The abutments 22 may be made symmetric, but are preferably asymmetric, with the proximal abutment (to the right-hand side as viewed) being closer to the base plate 12 than the distal abutment (to the left-hand side as viewed). This is because the longitudinal cut is preferably not made absolutely axially of the first metatarsal, as shown in FIG. 4.

A resilient restraint 36, for example, an elastic or rubber band is then used to secure the jig 10 to the foot 34, by looping the restraint 36 around one of the V shaped recesses 26, passing it around the upper part of the foot 34, wrapping it around the hook 30, and looping the other end around the other V shaped recess 26.

The jig 10 can also be secured further using pins or wires passed through the apertures 28, when the surgeon is satisfied that the jig selection and position is correct.

The surgeon is then able to rest a saw blade (not shown) on the planar surface 18 of the cutting guide 16, and to make a longitudinal cut through the first metatarsal 32 at the angle a relative to the plane of the base plate 12 and the plantar aspect of the foot 34. This cut is indicated at 38 in FIG. 4. Angle a determines the amount of vertical translation relative to the amount of lateral translation of the first metatarsal head. The positions of the ends of the cut 38 are controlled by the end stops 20, which limit the movement of the blade. The end stops 20 enable the surgeon, or operator, to make the distal and proximal limits of the cut 38 at a known angle with respect to the longitudinal axis of the cutting guide 16. Alternatively, in the absence of the stops 20, the abutments 22 act as stops.

In this embodiment, the proximal and distal transverse cuts of the osteotomy, indicated at 40 and 42 in FIG. 4 respectively, are cut freehand by the surgeon to align with the ends of the longitudinal cut 38, after removal of the jig.

A second embodiment of the invention will now be described with reference to FIG. 5 of the drawings. In this embodiment, the surgical jig 110 also comprises a base plate 112 having an upper reference surface 113, an arm 114 extending substantially perpendicularly from one end of the base plate 112, and a cutting guide or cutting block 116 mounted to the end of the arm 114.

The cutting guide 116 has a planar surface 118 disposed at an angle a relative to the reference surface 113 of the base plate 112. An appendage in the form of an abutment 122 extends from each end of the cutting guide 116 and is profiled on its edge with a substantially semi-circular recess 124, which, as in the first embodiment, is shaped to fit against an exposed metatarsal bone. The other side of the abutment is provided with hooks or lips 126. An aperture 128 is provided through each end of the cutting guide 116, through which a pin or wire can be passed in order to secure the cutting guide to the first metatarsal of the foot. A hook, not shown, similar to that of the first embodiment is provided on the underside of the base plate 112, i.e. extending substantially in a direction opposite to that of the arm 114.

There are no end stops as in the previous embodiment, which limit the movement of a cutting implement moving along the planar surface. Rather, the proximal and distal abutments 122 are disposed at the angle relative to the longitudinal axis of the cutting guide, and act as stops. Furthermore a cut-out or window 144 is provided in the arm 114 beneath the planar surface 118, which enables a surgeon to clearly see the position of the proximal transverse cut 40 of the scarf osteotomy. The cut out 144 extends through the cutting guide, so that the proximal transverse cut 40 can meet the longitudinal cut 38.

In use, the surgical jig 110 is used in the same way as the jig 10 of the first embodiment, save that the resilient restraint 36 is looped around the hooks or lips 126 of the abutments 122. Furthermore, the abutments 122 not only serve as stops, which limit the longitudinal cut 38, but also act as cutting guides for the proximal and distal transverse cuts 40,42, and are disposed at the angle relative to the longitudinal axis of the cutting guide 116.

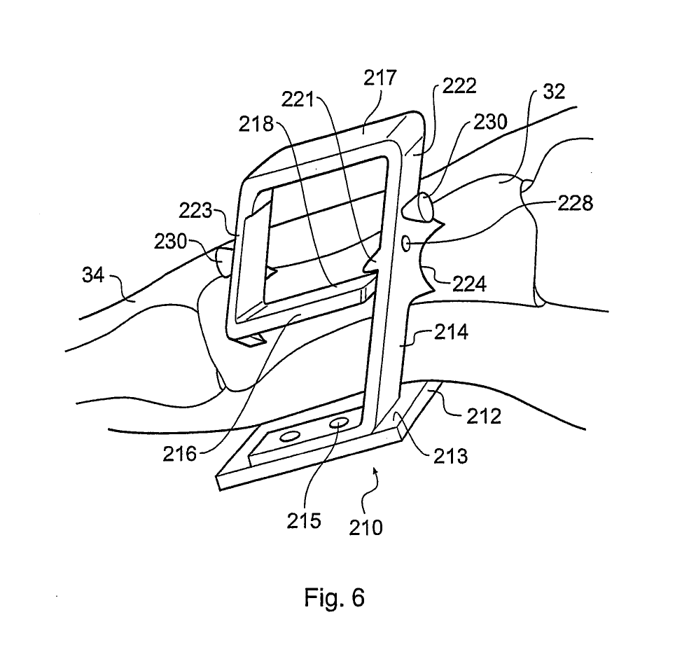

A third and most preferred embodiment of the invention will now be described with reference to FIG. 6 of the drawings. In this embodiment, as in the previous embodiments, the surgical jig 210 comprises a base plate 212 having an upper reference surface 213, an arm 214 extending substantially perpendicularly from one end of the base plate 212, and a cutting guide or cutting block 216 mounted to the end of the arm 214.

The arm 214 has an integral flange 215, which is riveted to the base plate 212. In other arrangements, the flange 215 may be attached to the base plate 212 by any suitable means, for example, welding or spot welding. The arm 214 extends away from the base plate 212 as a web, the plane of which lies substantially parallel with the longitudinal axis of the base plate 212. This is advantageous, because the arm 214 only minimally obscures the surgeon's view of the side of the foot 34, when the jig is in use. Furthermore, cutter access for making the proximal transverse cut 40 is unimpeded.

The arm 214 forms a proximal abutment 222, and a substantially horizontal member 217 extends from the upper end of the arm 214, as viewed, which terminates in a substantially downward member, forming a distal abutment 223. The cutting guide 216 has a planar surface 218, disposed at the angle a relative to the reference surface 213 of the base plate 212, and extends from the lower end of the distal abutment 223 back towards the arm 214, but does not meet it. In other words, the arm 214, the horizontal member 217, the downward member 223 and the cutting guide 216 substantially describe the shape of the numeral “9”, when viewed as in FIG. 6.

The abutments 222, 223 are profiled on their edges as in the previous embodiments with substantially semi-circular recesses 224, which are shaped to fit against an exposed metatarsal bone. An aperture 228 is provided through the arm 214, adjacent the recess 224, which may be used to secure the jig to the metatarsal as described above, by means of a pin or wire, or other suitable means. Conical members or hooks 230 are mounted to the outer edges of the abutments 222, 223, which allow for attachment of the resilient restraint 36, as described in the first two embodiments. In this regard, a hook, not shown, may be provided on the base plate 212.

The inner edges or faces of the abutments 222, 223 are disposed at the angle P relative to the longitudinal axis of the cutting guide, and act as stops for the longitudinal cut 38.

A further stop 221 is mounted to the inside edge of the arm 214 above the planar surface 218 of the cutting guide, which limits the proximal transverse cut as it meets the longitudinal cut 38.

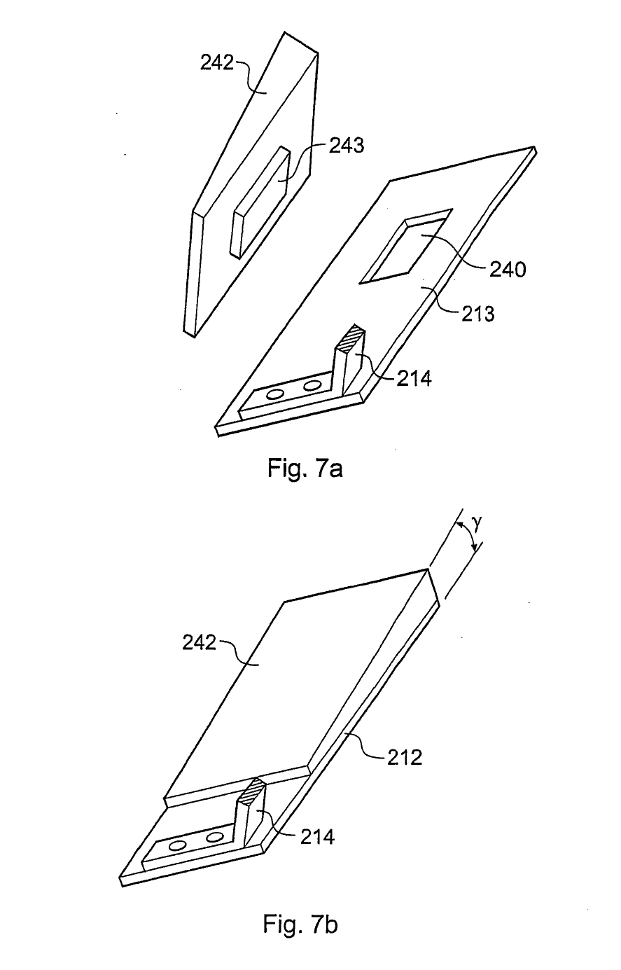

Referring also to FIGS. 7A and 7B, the base plate 212 has aperture 240 therethrough. A spacer 242, formed as a truncated wedge, has a projection 243 on one side thereof, which is shaped to be received with a push fit into the aperture 240, enabling the spacer to be mounted to the reference surface 213 of the base plate 212.

The upper surface of the spacer now forms the reference surface, which contacts the underside of, or the plantar aspect of, the forefoot 34. The angle of the wedge has the effect of increasing the angle a, and the greater the angle of the wedge, referenced y, the greater the angle a.

The use of the surgical jig 210 is substantially the same as that for the earlier jigs described. However, because of the shape of the jig 210, the operating area is clearly visible to the surgeon.

Each of the jigs 10, 110, 210 described can be manufactured from any suitable material, e.g., plastics or metal. The jigs are also intended to be manufactured as a set having different values of a and(3, such that the correct jig, or the most suitable jig can be selected according to a pre-operative plan. Furthermore, the jigs 10, 110, 210 can be used with any suitable type of surgical cutting tool, e.g., reciprocating or oscillating blades, lasers and water jets.

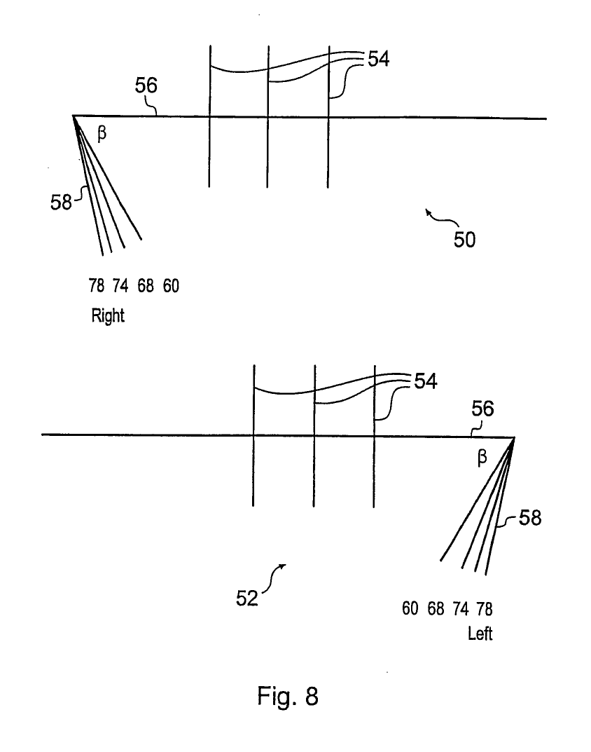

Referring now to FIG. 8, two templates 50, 52 are shown, for the right and left feet respectively. Each template 50, 52 comprises an overlay of visible indicia provided on a transparent sheet, for example, acetate. The indicia comprises three parallel lines 54, a central line 56 running perpendicularly through the three parallel lines 54, and a series of radiating lines 58 at the angle to the central line. These radiating lines each correspond to a jig in the set and represent the edge of the cutting guide.

Before operating, a weight-bearing antero-posterior X-ray film of the forefoot is taken, and the template for the appropriate foot is laid over the X-ray film. If the surgeon intends the length of the big toe to remain the same, the transverse cuts 40, 42 are to be perpendicular to the second metatarsal, the parallel lines 54 are aligned with the second metatarsal, and one of the radiating lines 56 placed adjacent the medial border of the first metatarsal. In other words, the angle (3 is approximately equal to the complementary angle of the IMA. The line 56 selected, corresponds to the jig to be used.

If the surgeon requires lengthening of the big toe, then a larger angle and associated jig is selected, but if the surgeon requires shortening of the big toe, then a smaller angle and associated jig is selected.

Although the jigs described herein are for use in treatment of Hallux Valgus, it should be appreciated that variations of these surgical jigs could be produced for other forefoot operations, the jigs having a reference member adapted to be placed against the plantar aspect of the foot. It is an advantage of jigs of the invention that by their use, consistency and repeatability of operations can be improved.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A surgical jig comprising:
a cutting guide adapted to guide a cutting implement; and
a reference surface adapted to be placed against the plantar aspect of a forefoot.

2. A surgical jig as claimed in claim 1, wherein the cutting guide includes a planar surface disposed at an angle to the reference surface.

3. A surgical jig as claimed in claim 1, wherein the reference surface is the upper surface of a base plate and the cutting guide is rigidly connected to the base plate in a fixed position.

4. A surgical jig as claimed in claim 3, wherein an angled spacer is provided for increasing the angle between the planar surface and the reference surface, the spacer being attached to the upper surface of the base plate, and the upper surface of the spacer becoming the reference surface.

5. A surgical jig as claimed in claim 2, wherein end stops are provided at one or both ends of the planar surface.

6. A surgical jig as claimed in claim 5, wherein the end stops are provided at a predetermined angle relative to a longitudinal axis of the cutting guide.

7. A surgical jig as claimed in claim 5, wherein the end stops are provided by abutments, the abutments having faces adjacent the planar surface and substantially perpendicular to a longitudinal axis of the cutting guide, the faces also serving as cutting guides.

8. A surgical jig as claimed in claim 7, wherein one of the faces extends from the upper side of the planar surface, and the other face extends from the lower side of the planar surface.

9. A surgical jig as claimed in claim 7, wherein one of the faces is spaced from an end of the planar surface, enabling the cutting implement to pass the planar surface.

10. A surgical jig as claimed in claim 7, wherein one or more stops are provided on the faces of the abutments.

11. A surgical jig as claimed in claim 10, wherein one of the stops is provided by the planar surface.

12. A surgical jig as claimed claim 7, wherein the abutments have a curved profile on one side, which is adapted to fit against an exposed metatarsal bone.

13. A surgical jig as claimed in claim 1, wherein a resilient retaining means (36) is provided to hold the surgical jig in a fixed position relative to the plantar aspect of the forefoot.

14. A surgical jig as claimed in claim 8, wherein one or more apertures are provided in the cutting guide, which enable the surgical jig to be pinned or wired to an exposed bone of the forefoot.

15. A surgical jig as claimed in claim 1, wherein the jig is one of a set of jigs, each having the cutting guide in a different orientation relative to the reference surface.

16. A surgical jig as claimed in claim 1, wherein a template is provided for selecting a surgical jig from the set of surgical jigs.

17. A surgical jig as claimed in claim 16, wherein the template comprises a transparent overlay, through which an X-ray can be viewed, the overlay having indicia thereon for comparing the orientation of the cutting guides of the jigs with the bones of the forefoot.

18. A surgical jig as claimed in claim 16, wherein the template comprises a transparent overlay, through which an X-ray can be viewed, the overlay having indicia thereon for comparing the predetermined angle relative to a longitudinal axis of the cutting guide of the jigs with the bones of the forefoot.

19. A template for selecting a surgical jig from a range of surgical jigs comprising:
a transparent overlay through which an X-ray can be viewed, the overlay having indicia thereon for comparing the orientation of cutting guides of the jigs with the bones of the forefoot.

20. A method of operating on a forefoot comprising the steps of:
(a) exposing a bone to be cut;
(b) referencing a surgical jig from the plantar aspect of the forefoot; and
(c) cutting a bone using the jig as a guide to guide a cutting tool.
